(19) United States
(12) Patent Application Publication
(10) Pub. No.: US 2012/0046483 A1
(43) Pub. Date: Feb. 23, 2012

(54) Catalyst for Esterification and Polyesterification, and Method for Making Esterified and Polyesterified Product by Using the Same

(75) Inventor: Shiue-Shien Weng, Kaohsiung (TW)

(73) Assignee: R.O.C. MILITARY ACADEMY, Kaohsiung (TW)

(21) Appl. No.: 12/858,024

(22) Filed: Aug. 17, 2010

(51) Int. Cl. C07F 15/02; C07C 67/02

(52) U.S. Cl. 556/146; 560/1

(57) ABSTRACT

A catalyst for esterification and polyesterification is proposed. The catalyst is an iron acetylacetonate complex having a formula of Fe(RCOCH₂COR)₃, where R is methyl or a substituent other than methyl. A method for making an esterified product or a polyesterified product by using the iron acetylacetonate complex through esterification and polyesterification is also disclosed so as to improve the existing processes where hydrochloric acid or sulfuric acid used as the catalyst causes yellowed products, danger operation and environmental pollution.

### BACKGROUND OF THE INVENTION

1. Technical Field

The present invention relates to catalysts for esterification and polyesterification. More particularly, the present invention relates to a complex acting in an esterification reaction or a polyesterification reaction as a catalyst. The present invention also relates to a method for making an esterified or polyesterified product by using the complex in an esterification reaction or a polyesterification reaction as a catalyst.

2. Description of Related Art

Esterification is generally known as a reversible reaction. In the conventional esterification process performed in laboratories and factories, an acid and an alcohol are refluxed in the presence of a strong acid as a catalyst, such as anhydrous hydrochloric acid (HCl) or sulfuric acid (H₂SO₄). As the foregoing reaction is an equilibrium reaction, for ensuring the equilibrium moving toward the desired product, anhydrous hydrochloric acid or concentrated sulfuric acid has to be used as a catalyst or drying agent. However, the strong acid brings significant concerns about toxicity and danger in addition to the increased manufacturing cost. Besides, the exothermic reaction, as a potential result of the event that water freed by the strong acid re-contacts the excessive strong acid, may lead to fire or even explosions, which are highly dangerous, not to mention a pollution to the environment. Moreover, as strong acids are known to decompose functional groups in many organic compounds, the aforementioned process is limited to the esterification for simple alkyl acids, and inapplicable for many special molecules for industries, functionalized polymers and fine chemical molecules. Furthermore, as it is known that the catalyst influences the product of the esterification in color, the effective catalyst for polyester products (e.g., polyethylene terephthalate) is likely to lead to unacceptable yellowed products, which need additional time and costs to be bleached, causing the esterification uneconomical and inconvenient.

Relatively successful catalysis systems that have been developed thereafter include Lewis acid-catalyzed systems and

Lewis base-catalyzed systems. In the former group, organotin compounds and other compounds such as antimony, titanium and lithium are used as the catalyst. Therein, while antimony, titanium and lithium are commercially available, they are sensitive to the air and thus seriously need to be used with anhydrous solvents in the esterification. Besides, since the above-named compounds all contain rare metals, they are expensive in cost and inferior in operational practicability. As to organotin compounds, they are highly toxic and carcinogenic, while tending to remain on the products of the esterification, thus being dangerous to humans. Additionally, the esterification using organotin compounds as the catalyst needs to be performed under high temperature (about 120° C. to about 150° C.) and this also leads to an increase of costs. In Lewis base-catalyzed systems, metal alkoxide complexes such as sodium alcoholate and aluminum alcoholates are used as the catalyst. While being relatively inexpensive in cost and highly air-stable, metal alkoxide complexes are of strong base. This causes them inapplicable to those molecules for industries and functionalized polymers that are base-sensitive and significantly limited in regard to their applications.

In addition, the reaction solvent for esterification is conventionally benzene or toluene. Toluene is of strong toxicity, so it is harmful to the human body exposed therein and pollutant to the environment. Also, the traditional catalysts for esterification are hardly recycled. All these defects suggest the need to improve the existing processes for esterification.

### SUMMARY OF THE INVENTION

The primary objective of the present invention is to provide improvement to esterification and polyesterification by proposing an iron acetylacetonate complex, composed of a trivalent ferric ion (Fe³⁺) and acetyl acetone, as a catalyst. Since acetyl acetone having high coordination property serves to stabilize the trivalent ferric ion, the iron acetylacetonate complex is highly active and reactive while being very stable in contact with the air and water and resistant to room-temperature oxidation and room-temperature deterioration. The iron acetylacetonate complex is nontoxic so it is safe to the human body and the environment. By using the iron acetylacetonate complex, the present invention improves the existing processes for esterification and polyesterification where hydrochloric acid or sulfuric acid as the catalyst causes yellowed products, danger operation and environmental pollution.

For achieving the above objective, the present invention proposes a catalyst for esterification and polyesterification, wherein the catalyst is an iron acetylacetonate complex that has a formula of Fe(RCOCH₂COR)₃, where R is methyl or a substituent other than methyl.

For achieving the above objective, the present invention further proposes a method for making an esterified product or a polyesterified product by using the above-mentioned iron acetylacetonate complex as a catalyst in an esterification reaction or a polyesterification reaction. The method includes mixing an ester and an alcohol to be reacted in a reaction solvent, adding the catalyst in the reaction solvent to form a mixture and heating the mixture to a reaction temperature. The method is characterized in that the iron acetylacetonate complex as the catalyst has a formula of Fe(RCOCH₂COR)₃, where R is methyl or a substituent other than methyl.

### BRIEF DESCRIPTION OF THE DRAWINGS

FIG. 1 is a graph of chemical structure of a catalyst according to the present invention.

FIG. 2 is a flowchart of an esterification or polyesterification reaction using the catalyst of FIG. 1 according to the present invention.

FIG. 3 is a graph of chemical structure of the catalyst with methyl substituent replaced by trifluoromethyl (CF₃).

FIG. 4 is a graph of chemical structure of the catalyst with methyl substituent replaced by phenyl (Ph).

FIG. 5 is a graph of chemical structure of the catalyst with methyl substituent replaced by n-butyl.

### DETAILED DESCRIPTION OF THE INVENTION

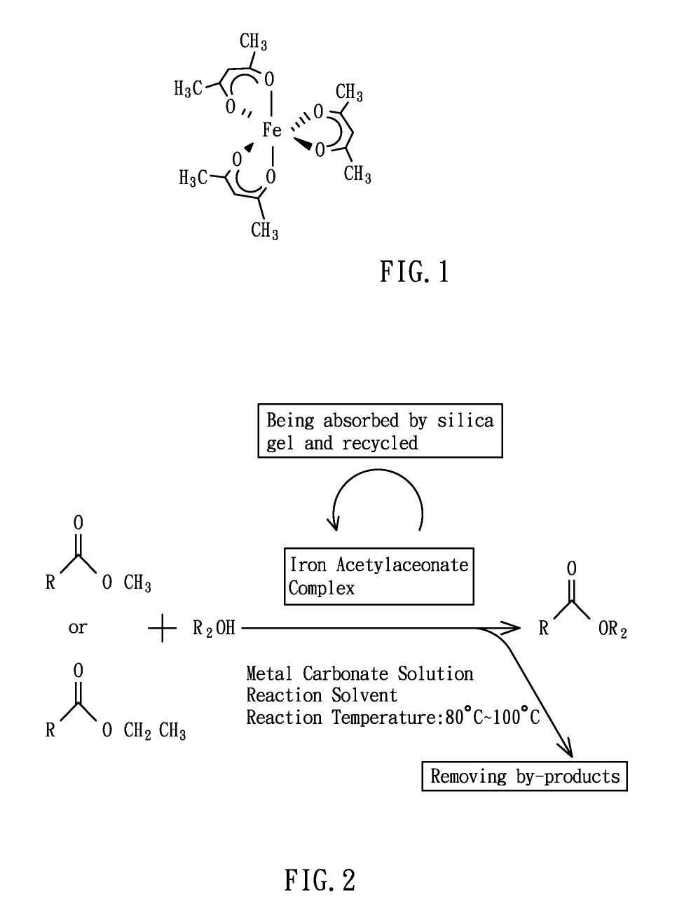

Referring to FIG. 1 and FIG. 2, a catalyst for esterification and polyesterification is proposed by the present invention. The catalyst is an iron acetylacetonate complex. In esterification or polyesterification, an ester and an alcohol to be reacted are added into a reaction solvent. The reaction solvent is n-heptane. Then an iron acetylacetonate complex is added as a catalyst. The iron acetylacetonate complex has a formula of Fe(RCOCH₂COR)₃, where R is methyl or a substituent other than methyl.

The present invention also proposes a method making an esterified product or a polyesterified product by using the iron acetylacetonate complex as a catalyst in an esterification reaction or a polyesterification reaction. The method involves mixing an ester and an alcohol to be reacted in a reaction solvent, adding the catalyst, and heating the above to a reaction temperature, which is between about 80° C. and about 100° C., so as to make the ester and the alcohol perform reaction in the presence of the catalyst, and thereby produce the esterified or polyesterified product. The reaction solvent may further contain 5 mol % of a metal carbonate solution (e.g., a sodium carbonate solution) for prompting the reaction. After the reaction, silica gel may be used to absorb the catalyst so as to recycle the catalyst for reuse.

In another embodiment, the aforementioned esterification and polyesterification include making a functionalized methyl or an ethyl ester react with a functionalized alcohol in the presence of the catalyst, so as to prepare the product.

Referring to FIG. 2, the present invention uses a complex of trivalent ferric acetyl acetone as a catalyst for esterification and polyesterification, and thus only 5 mol % of the catalyst is sufficient for transesterification reaction of various acid-sensitive esters, base-sensitive esters, alkyl esters, aryl esters, ether-containing esters, acid group-containing esters and fatty acid esters that contain long-chain cis-double bonds. For reaction, 5 mol % of the iron acetylacetonate complex is used as the catalyst, and the functionalized methyl or the ethyl ester are mixed with the functionalized methyl or the ethyl alcohol in a flask. Nontoxic n-heptane is used as the required solvent and may be added with 5 mol % of a metal carbonate, such as sodium carbonate (Na₂CO₃), potassium carbonate (K₂CO₃) or caesium carbonate (Cs₂CO₃) for accelerating the esterification and polyesterification. The mixture of the above is then heated to a reaction temperature for refluxing the solvent, wherein the reaction temperature ranges between about 80° C. and about 100° C. During the reaction, a dehydrating means is applied to remove excessive water freed by the reaction, while the by-products of the reaction such as methanol or ethanol are absorbed by a molecular sieve according to Soxhlet extraction method that acts as an alcohol-based adsorbent, so as to ensure the by-products of the reaction are absent from the resultant esterified product, thereby obtaining the esterified product of the present invention. The catalyst can be absorbed by silica gel and washed by ethanol containing 1% of acetyl acetone for reuse.

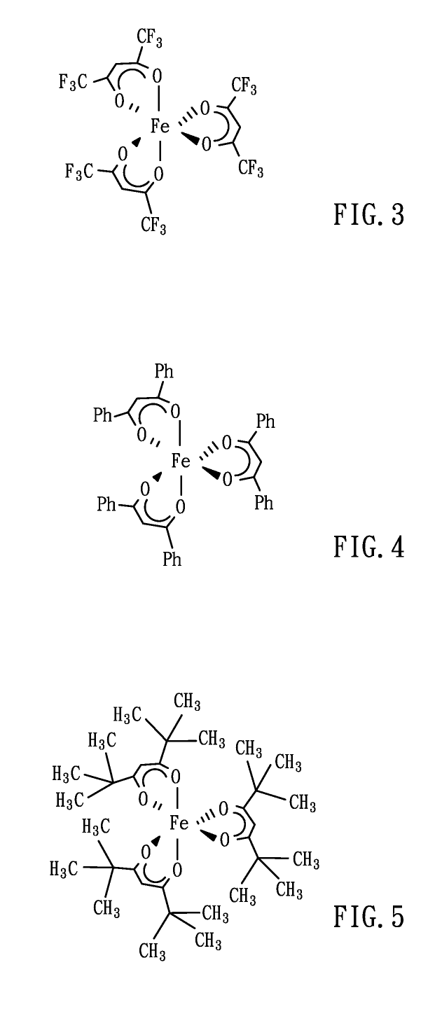

Referring to FIG. 3 through FIG. 5, there are additional embodiments of the iron acetylacetonate complex having the formula of Fe(RCOCH₂COR)₃, where R is methyl or a substituent other than methyl. While R in the embodiment of FIG. 1 is methyl, the catalyst for esterification and polyesterification may be one of the iron acetylacetonate complexes shown in FIG. 3 through FIG. 5 or analogues thereof. Therefore, in the additional embodiments, methyl in the iron acetylacetonate complex may be replaced by trifluoromethyl, so the catalyst is a trifluoromethyl iron acetylacetonate complex, replaced by phenyl, so the catalyst is a dibenzoyl methyl iron acetylacetonate complex, or replaced by n-butyl, so the catalyst is a 2,2,6,6-tetramethyl-3,5-dioxoheptane iron acetylacetonate complex.

The present invention has been described with reference to the preferred embodiments and it is understood that the embodiments are not intended to limit the scope of the present invention. Moreover, as the contents disclosed herein should be readily understood and can be implemented by a person skilled in the art, all equivalent changes or modifications which do not depart from the concept of the present invention should be encompassed by the appended claims.

What is claimed is:

1. A catalyst for an esterification reaction or a polyesterification reaction, the catalyst being an iron acetylacetonate complex having a formula of Fe(RCOCH₂COR)₃, where R is methyl or a substituent other than methyl.

2. The catalyst of claim 1, wherein the substituent is trifluoromethyl (CF₃) and the catalyst is a trifluoromethyl iron acetylacetonate complex.

3. The catalyst of claim 1, wherein the substituent is phenyl (Ph) and the catalyst is a dibenzoyl methyliron acetylacetonate complex.

4. The catalyst of claim 1, wherein the substituent is n-butyl and the catalyst is a 2,2,6,6-tetramethyl-3,5-dioxoheptane iron acetylacetonate complex.

5. A method of making an esterified product or a polyesterified product by using an iron acetylacetonate complex as a catalyst in an esterification reaction or a polyesterification reaction, the method including mixing an ester and an alcohol to be reacted in a reaction solvent, adding the catalyst in the reaction solvent to form a mixture, and heating the mixture to a reaction temperature, wherein the iron acetylacetonate complex has a formula of Fe(RCOCH₂COR)₃, where R is methyl or a substituent other than methyl.

6. The method of claim 5, wherein the reaction temperature is between about 80° C. and about 100° C.

7. The method of claim 5, wherein the reaction solvent is n-heptane and contains 5 mol % of a metal carbonate solution for accelerating the esterification reaction or the polyesterification reaction.

8. The method of claim 5, wherein the catalyst is absorbed by silica gel after the esterification reaction or the polyesterification reaction so as to recycle the catalyst for reuse.

9. The method of claim 5, wherein the ester is a functionalized ester and the alcohol is a functionalized alcohol.
